(19) United States
(12) Patent Application Publication
(10) Pub. No.: US 2020/0205714 A1
(43) Pub. Date: Jul. 2, 2020

(54) IMPRESSION MEASUREMENT DEVICE AND NON-TRANSITORY COMPUTER READABLE MEDIUM

(71) Applicant: FUJI XEROX CO., LTD., Tokyo (JP)

(72) Inventor: Hideto YUZAWA, Kanagawa (JP)

(73) Assignee: FUJI XEROX CO., LTD., Tokyo (JP)

(21) Appl. No.: 16/540,063

(22) Filed: Aug. 14, 2019

(30) Foreign Application Priority Data
Dec. 28, 2018 (JP) 2018-248476

(51) Int. Cl. A61B 5/16; A61B 5/024; G06F 16/901; G06F 16/2457

(52) U.S. Cl. CPC A61B 5/165; G06F 16/24578; G06F 16/9024; A61B 5/02405

(57) ABSTRACT

An impression measurement device is provided with an acquisition unit that, in a case in which a user interviews multiple persons in an order, acquires biological information of the user during the interview with each of the multiple persons, a setting unit that, in a case of deciding weight values assigned to the biological information acquired by the acquisition unit for each of the multiple persons, sets larger weight values for later persons compared to earlier persons in the order, and a correction unit that uses the weight values set by the setting unit to correct the biological information of the user with respect to each of the multiple persons.

### CROSS-REFERENCE TO RELATED APPLICATIONS

This application is based on and claims priority under 35 USC 119 from Japanese Patent Application No. 2018-248476 filed Dec. 28, 2018.

### BACKGROUND

### (i) Technical Field

The present disclosure relates to an impression measurement device and a non-transitory computer readable medium.

### (ii) Related Art

For example, Japanese Unexamined Patent Application Publication No. H11-004892 describes a biological attunement detection device that objectively enables deeper recognition of a psychological negotiation with a partner in a dialogue or communication using a communicating means. The biological attunement detection device is provided with a biological information detecting means that detects biological information, a biological information receiving means that receives biological information, an attunement determining means that compares the biological information detected by the biological information detecting means and the biological information received by the biological information receiving means, and determines the attunement between the two, and a notifying means that issues a notification of the determination result of the attunement determining means.

### SUMMARY

Meanwhile, when measuring the impression of a partner using biological information, in a situation of interviewing multiple partners in order, the biological response gradually becomes duller due to fatigue and the like, making it difficult to measure the impression of partners later in the order in some cases.

Aspects of non-limiting embodiments of the present disclosure relate to providing, when a user interviews multiple persons in an order, an impression measurement device and a non-transitory computer readable medium capable of accurately measuring the impression of a partner compared to a case in which the impression of the partner is influenced by the interview order.

Aspects of certain non-limiting embodiments of the present disclosure address the above advantages and/or other advantages not described above. However, aspects of the non-limiting embodiments are not required to address the advantages described above, and aspects of the non-limiting embodiments of the present disclosure may not address advantages described above.

According to an aspect of the present disclosure, there is provided an impression measurement device provided with an acquisition unit that, in a case in which a user interviews multiple persons in an order, acquires biological information of the user during the interview with each of the multiple persons, a setting unit that, in a case of deciding weight values assigned to the biological information acquired by the acquisition unit for each of the multiple persons, sets larger weight values for later persons compared to earlier persons in the order, and a correction unit that uses the weight values set by the setting unit to correct the biological information of the user with respect to each of the multiple persons.

### BRIEF DESCRIPTION OF THE DRAWINGS

Exemplary embodiments of the present disclosure will be described in detail based on the following figures, wherein:

FIG. 1 is a diagram illustrating one example of a configuration of an impression measurement system according to a first exemplary embodiment;

FIG. 2 is a block diagram illustrating one example of an electrical configuration of an impression measurement device according to the first exemplary embodiment;

FIG. 3 is a graph illustrating one example of biological information according to the first exemplary embodiment;

FIG. 4 is a block diagram illustrating one example of a functional configuration of the impression measurement device according to the first exemplary embodiment;

FIG. 5A is a graph illustrating one example of biological information before correction according to an exemplary embodiment;

FIG. 5B is a graph illustrating one example of corrected biological information according to an exemplary embodiment;

FIG. 6A is a diagram illustrating one example of a correction table according to an exemplary embodiment;

FIG. 6B is a diagram accompanying a description of the correction of biological information according to an exemplary embodiment;

FIG. 7 is a flowchart illustrating one example of the flow of a process by an impression measurement processing program according to the first exemplary embodiment;

FIG. 8A is a graph illustrating one example of biological information of a user according to an exemplary embodiment;

FIG. 8B is a diagram illustrating one example of a biological data table according to an exemplary embodiment;

FIG. 9 is a diagram illustrating one example of a difference data table according to an exemplary embodiment;

FIG. 10 is a diagram illustrating one example of a weight value table according to an exemplary embodiment;

FIG. 11 is a diagram illustrating one example of a corrected data table according to an exemplary embodiment;

FIG. 12 is a graph illustrating one example of corrected biological information according to an exemplary embodiment;

FIG. 13A is a graph illustrating another example of biological information of a user according to an exemplary embodiment;

FIG. 13B is a diagram illustrating another example of a biological data table according to an exemplary embodiment;

FIG. 14 is a diagram illustrating another example of a weight value table according to an exemplary embodiment;

FIG. 15 is a diagram accompanying a description of data compression according to an exemplary embodiment;

FIG. 16 is a graph illustrating another example of corrected biological information according to an exemplary embodiment;

FIG. 17 is a graph illustrating one example of biological information according to a second exemplary embodiment;

FIG. 18 is a block diagram illustrating one example of a functional configuration of the impression measurement device according to the second exemplary embodiment;

FIG. 19 is a flowchart illustrating one example of the flow of a process by an impression measurement processing program according to the second exemplary embodiment;

FIG. 20 is a graph illustrating one example of corrected biological information according to an exemplary embodiment;

FIG. 21 is a diagram illustrating one example of a slope table and a rank score table according to an exemplary embodiment;

FIG. 22 is a diagram illustrating one example of a primary slope score according to an exemplary embodiment;

FIG. 23 is a diagram illustrating one example of a secondary slope score according to an exemplary embodiment;

FIG. 24 is a diagram illustrating one example of a primary interview order score according to an exemplary embodiment;

FIG. 25 is a diagram illustrating one example of a secondary interview order score according to an exemplary embodiment;

FIG. 26 is a diagram illustrating one example of a total score according to an exemplary embodiment;

FIG. 27 is a diagram illustrating one example of a rank table according to an exemplary embodiment;

FIG. 28 is a graph illustrating another example of corrected biological information according to an exemplary embodiment;

FIG. 29 is a diagram illustrating another example of a slope table and a rank score table according to an exemplary embodiment;

FIG. 30 is a diagram illustrating another example of a primary slope score according to an exemplary embodiment;

FIG. 31 is a diagram illustrating another example of a secondary slope score according to an exemplary embodiment;

FIG. 32 is a diagram illustrating another example of a primary interview order score according to an exemplary embodiment;

FIG. 33 is a diagram illustrating another example of a secondary interview order score according to an exemplary embodiment;

FIG. 34 is a diagram illustrating another example of a total score according to an exemplary embodiment;

FIG. 35 is a diagram illustrating another example of a rank table according to an exemplary embodiment;

FIG. 36 is a graph illustrating another example of corrected biological information according to an exemplary embodiment;

FIG. 37 is a diagram illustrating another example of a slope table and a rank score table according to an exemplary embodiment;

FIG. 38 is a diagram illustrating another example of a primary slope score according to an exemplary embodiment;

FIG. 39 is a diagram illustrating another example of a secondary slope score according to an exemplary embodiment;

FIG. 40 is a diagram illustrating another example of a primary interview order score according to an exemplary embodiment;

FIG. 41 is a diagram illustrating another example of a secondary interview order score according to an exemplary embodiment;

FIG. 42 is a diagram illustrating another example of a total score according to an exemplary embodiment;

FIG. 43 is a diagram illustrating another example of a rank table according to an exemplary embodiment; and

FIG. 44 is a graph illustrating another example of biological information before correction according to an exemplary embodiment.

### DETAILED DESCRIPTION

Hereinafter, exemplary embodiments for carrying out the present disclosure will be described in detail and with reference to the drawings.

### First Exemplary Embodiment

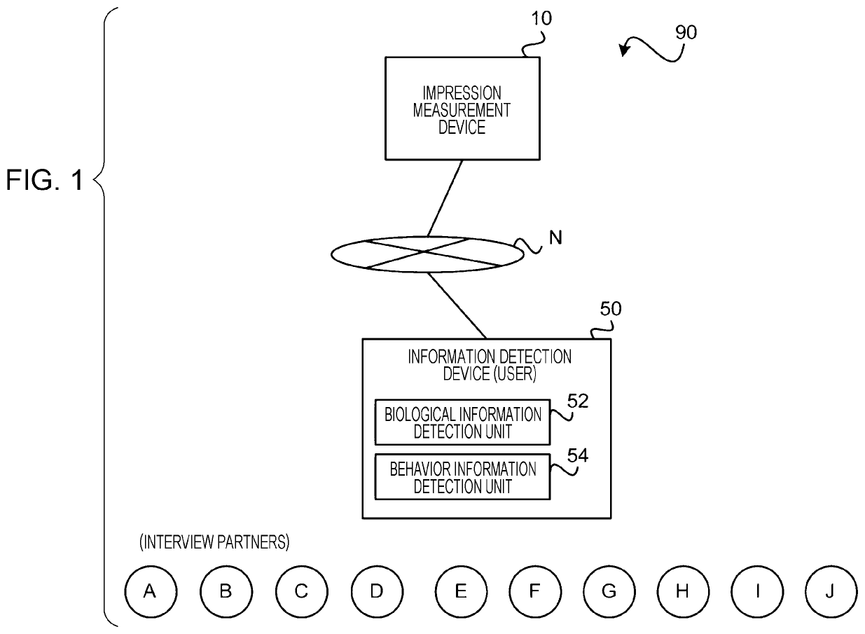

FIG. 1 is a diagram illustrating one example of a configuration of an impression measurement system 90 according to the first exemplary embodiment.

As illustrated in FIG. 1, the impression measurement system 90 according to the present exemplary embodiment is provided with an impression measurement device 10 and an information detection device 50. For the impression measurement device 10, a general-purpose computer device such as a server computer or a personal computer (PC) is applied, for example.

The impression measurement device 10 according to the present exemplary embodiment is connected to the information detection device 50 via a network N. Note that for the network N, a network such as the Internet, a local area network (LAN), or a wide area network (WAN) is applied, for example.

The information detection device 50 according to the present exemplary embodiment is provided with a biological information detection unit 52 and a behavior information detection unit 54. The biological information detection unit 52 detects biological information about a user to be measured (measuree), and transmits the detected biological information to the impression measurement device 10. The information used as the biological information referred to herein is, for example, a component that varies at a high frequency corresponding to respiratory variation (hereinafter called the “high-frequency (HF) component”), a component that varies at a low frequency corresponding to blood pressure variation (hereinafter called the “low-frequency (LF) component”), and the combined total of the HF component and the LF component (hereinafter called the “total power (TP)”). More specifically, the HF component is a varying wave whose signal source is respiration, which has a period from three to four seconds approximately, while the LF component is a varying wave whose signal source is blood pressure changes, which have a period of approximately 10 seconds called Mayer waves.

It has been established that the magnitudes expressed in the HF component and the LF component change depending on the balance of tension between the sympathetic nervous system and the parasympathetic nervous system. This property is utilized to estimate the balance of the autonomic nervous system from the HF component and the LF component. Typically, the HF component is treated as an index indicating the working of the parasympathetic nervous system, while the LF component is treated as an index indicating the working of the sympathetic nervous system. The combined total TP of the LF component and the HF component is treated as an index indicating the working of autonomic nerve function, and is also called the autonomic nerve activity.

The LF component and the HF component are computed by performing frequency analysis (time-frequency analysis) on time-series data such as heartbeat variation, pulse variation, and acceleration pulse wave variation. Established methods of the related art are applicable as the methods of the frequency analysis, and for example, the maximum entropy method (MEM), the fast Fourier transform (FFT) method, or the wavelet method is applied.

On the other hand, the behavior information detection unit 54 detects behavior information about the user to be measured, and transmits the detected behavior information to the impression measurement device 10. The behavior information will be described later.

In a case in which the user to be measured interviews multiple persons (in the example illustrated in FIG. 1, ten persons from A to J) in order, the impression measurement device 10 according to the present exemplary embodiment is provided with a function of using the biological information about the user to measure an impression of each of the interview partners (A to J).

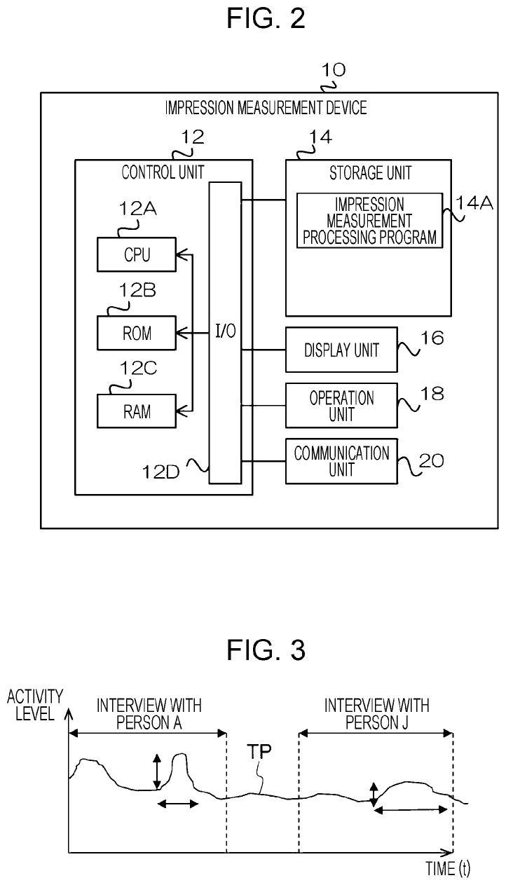

FIG. 2 is a block diagram illustrating one example of an electrical configuration of the impression measurement device 10 according to the first exemplary embodiment.

As illustrated in FIG. 2, the impression measurement device 10 according to the present exemplary embodiment is provided with a control unit 12, a storage unit 14, a display unit 16, an operation unit 18, and a communication unit 20.

The control unit 12 is provided with a central processing unit (CPU) 12A, read-only memory (ROM) 12B, random access memory (RAM) 12C, and an input/output interface (I/O) 12D, which are interconnected via a bus.

Each functional unit, including the storage unit 14, the display unit 16, the operation unit 18, and the communication unit 20, is connected to the I/O 12D. Each of these functional units is capable of bidirectional communication with the CPU 12A via the I/O 12D.

The control unit 12 may be configured as a sub-controller that controls a subset of operations of the impression measurement device 10, or may be configured as a main controller that controls all operations of the impression measurement device 10. An integrated circuit such as a large-scale integration (LSI) chip or an integrated circuit (IC) chipset is used for some or all of the blocks of the control unit 12, for example. A discrete circuit may be used for each of the above blocks, or a circuit integrating some or all of the above blocks may be used. The above blocks may be provided together as a single body, or some blocks may be provided separately. Also, a part of each of the above blocks may be provided separately. The integration of the control unit 12 is not limited to LSI, and a dedicated circuit or a general-purpose processor may also be used.

For the storage unit 14, a hard disk drive (HDD), a solid-state drive (SSD), flash memory, or the like is used, for example. In the storage unit 14, an impression measurement processing program 14A for realizing an impression measurement process according to the present exemplary embodiment is stored. Note that the impression measurement processing program 14A may also be stored in the ROM 12B.

The impression measurement processing program 14A may be preinstalled in the impression measurement device 10, for example. The impression measurement processing program 14A may also be realized by being stored on a non-volatile storage medium or distributed over the network N and appropriately installed in the impression measurement device 10. Note that anticipated examples of the non-volatile storage medium include a Compact Disc—Read-Only Memory (CD-ROM), a magneto-optical disc, an HDD, a Digital Versatile Disc—Read-Only Memory (DVD-ROM), flash memory, a memory card, and the like.

For the display unit 16, a liquid crystal display (LCD), an organic electroluminescence (EL) display, or the like is used, for example. The display unit 16 may include an integrated touch panel. The operation unit 18 is provided with a device for performing input operations, such as a keyboard and mouse, for example. The display unit 16 and the operation unit 18 receive various instructions from a measurer of the impression measurement device 10. The display unit 16 displays various information such as the results of processes executed according to instructions received from the measurer and notifications about processes.

The communication unit 20 is connected to the network N such as the Internet, a LAN, or a WAN, and is capable of communicating with the information detection device 50 over the network N.

FIG. 3 is a graph illustrating one example of biological information according to the first exemplary embodiment.

In FIG. 3, the vertical axis indicates activity level while the horizontal axis indicates time. The biological information illustrated in FIG. 3 is the TP detected from a user who interviews the multiple persons (A to J) illustrated in FIG. 1 in order. Note that the activity level referred herein represents the magnitude of the TP. In the present exemplary embodiment, a larger TP activity level (vertical component) or a shorter TP response time (horizontal component) expresses a better (more favorable) impression.

When utilizing the above biological information to measure the impression of a partner, in a situation of interviewing from the person A to the person J in order, the biological responses of the user gradually become duller due to fatigue and the like. In other words, as illustrated in FIG. 3, even if the user has a favorable impression of the person J that is similar to the impression of the person A, since the person J is interviewed later than the person A and the TP of the user has become dull, it is difficult to measure the impression accurately for a partner later in the order in some cases.

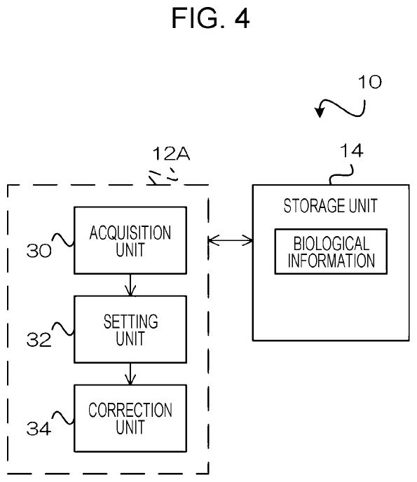

For this reason, by having the CPU 12A of the impression measurement device 10 according to the present exemplary embodiment load the impression measurement processing program 14A stored in the storage unit 14 into the RAM 12C and execute the impression measurement processing program 14A, the CPU 12A functions as each unit illustrated in FIG. 4.

FIG. 4 is a block diagram illustrating one example of a functional configuration of the impression measurement device 10 according to the first exemplary embodiment.

As illustrated in FIG. 4, the CPU 12A of the impression measurement device 10 according to the present exemplary embodiment functions as an acquisition unit 30, a setting unit 32, and a correction unit 34.

As one example, in the case where the user interviews multiple persons in order as illustrated in FIG. 1 described above, the acquisition unit 30 according to the present exemplary embodiment acquires biological information of the user during the interview with each of the multiple persons. Note that the interview time per person is associated with the biological information in advance. With this arrangement, the order in the biological information is specified. Also, as one example, the biological information is expressed by the relationship between each of the multiple persons and the TP.

In the case of deciding weight values assigned to the biological information acquired by the acquisition unit 30 for each of the multiple persons, the setting unit 32 according to the present exemplary embodiment sets larger weight values for later persons compared to earlier persons in the interview order.

The correction unit 34 according to the present exemplary embodiment uses the weight values set by the setting unit 32 to correct the biological information of the user with respect to each of the multiple persons.

Specifically, the setting unit 32 sets a weight value with respect to the TP activity level. In this case, the correction unit 34 corrects the biological information such that the activity level becomes larger.

Also, the setting unit 32 sets a weight value with respect to the TP response time. In this case, the correction unit 34 corrects the biological information such that the response time becomes shorter.

Also, the setting unit 32 sets a weight value with respect to each of the TP activity level and the TP response time. In this case, the correction unit 34 corrects the biological information such that the activity level becomes larger and the response time becomes shorter.

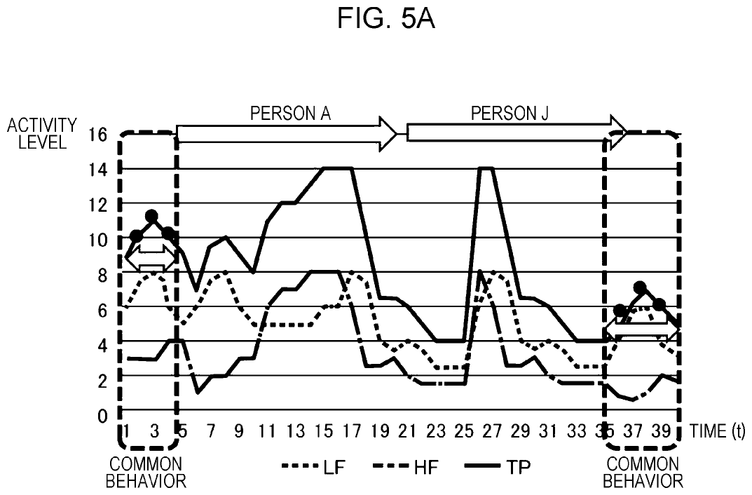

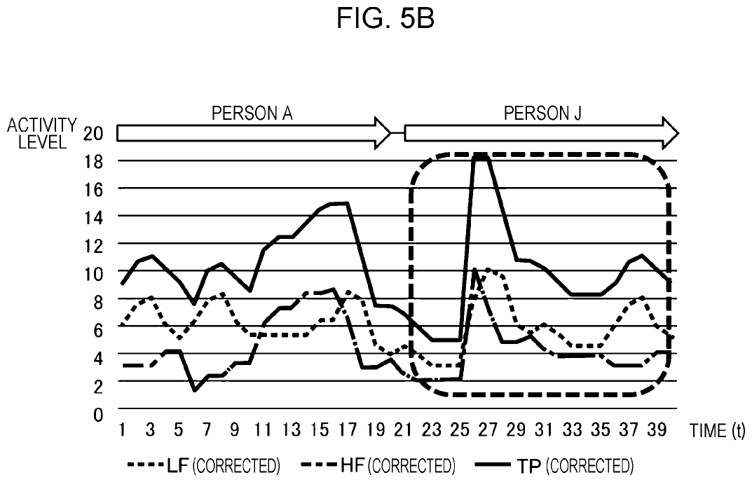

FIG. 5A is a graph illustrating one example of biological information before correction according to the present exemplary embodiment.

FIG. 5B is a graph illustrating one example of corrected biological information according to the present exemplary embodiment.

In FIGS. 5A and 5B, the vertical axis indicates activity level while the horizontal axis indicates time. The dashed line indicates the LF component, the chain line indicates the HF component, and the solid line indicates the TP.

The weight values described above are set on the basis of biological information corresponding to each of a common behavior performed before and after the interviews, and the interview order, as illustrated in FIG. 5A as one example. As one example, the common behavior referred to herein includes behaviors such as common greetings (for example, sitting down, then standing up, then sitting down) performed before and after each interview. Also, the biological information is corrected using the weight values such that the activity level becomes larger and the response time becomes shorter, as illustrated in FIG. 5B as one example.

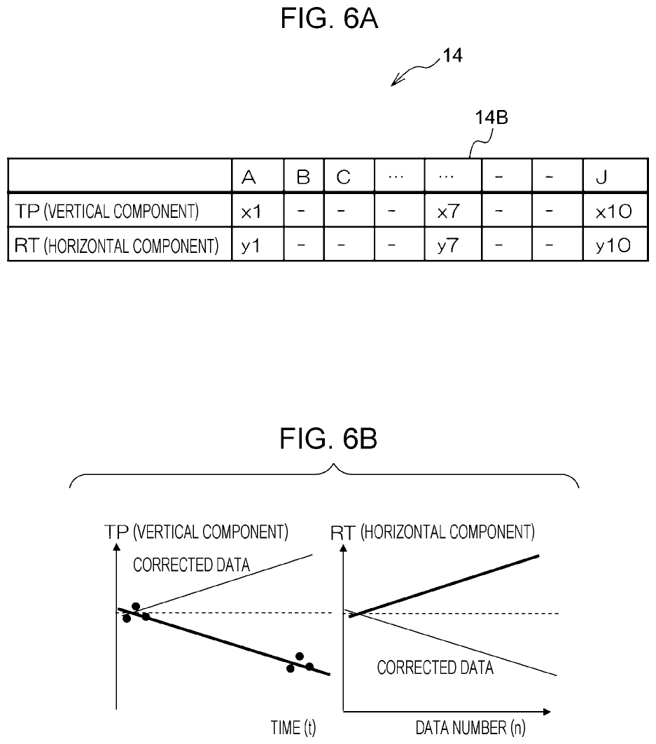

FIG. 6A is a diagram illustrating one example of a correction table 14B according to the present exemplary embodiment.

In the correction table 14B illustrated in FIG. 6A, a correction coefficient for each of the activity level TP (vertical component) and the response time RT (horizontal component) is set for each of the multiple persons (herein, ten persons from A to J). The correction table 14B is stored in the storage unit 14.

The correction coefficients set in the correction table 14B illustrated in FIG. 6A are coefficients derived on the basis of the weight values described above. The activity level TP correction coefficients (x1 to x10) are set to be larger for partners later in the order, while the response time RT correction coefficients (y1 to y10) are set to be smaller for partners later in the order. Note that a specific method of deriving the weight values will be described later.

FIG. 6B is a diagram accompanying a description of the correction of biological information according to the present exemplary embodiment.

In the diagram on the left in FIG. 6B, the vertical axis indicates activity level TP (vertical component) while the horizontal axis indicates time. In the diagram on the right in FIG. 6B, the vertical axis indicates response time RT (horizontal component) while the horizontal axis indicates a data number (herein, n=10). The data number corresponds to the interview partners, in which the data number of the person A is 1 and the data number of the person J is 10.

As illustrated in the diagram on the left in FIG. 6B, in the case of supposing that the activity level TP varies linearly, the activity level TP (thick line) decreases with the passage of time, but by using the correction table 14B illustrated in FIG. 6A, the activity level TP (thin line) is corrected to increase with the passage of time. Also, as illustrated in the diagram on the right in FIG. 6B, in the case of supposing that the response time RT varies linearly, the response time RT (thick line) increases as the data number increases, but by using the correction table 14B illustrated in FIG. 6A, the response time RT (thin line) is corrected to decrease as the data number increases.

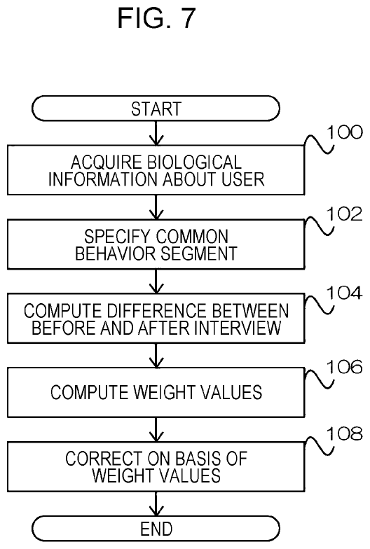

Next, FIGS. 7 to 12 will be referenced to describe the action of the impression measurement device 10 according to the first exemplary embodiment. Herein, the case of correcting the TP activity level to become larger will be described.

FIG. 7 is a flowchart illustrating one example of the flow of a process by the impression measurement processing program 14A according to the first exemplary embodiment.

First, if the impression measurement device 10 is instructed by a person in charge of measurement to launch the impression measurement processing program 14A, each of the following steps is executed. Note that biological information of the user when interviewing multiple persons in order (herein, ten persons from the person A to the person J) is stored in the storage unit 14.

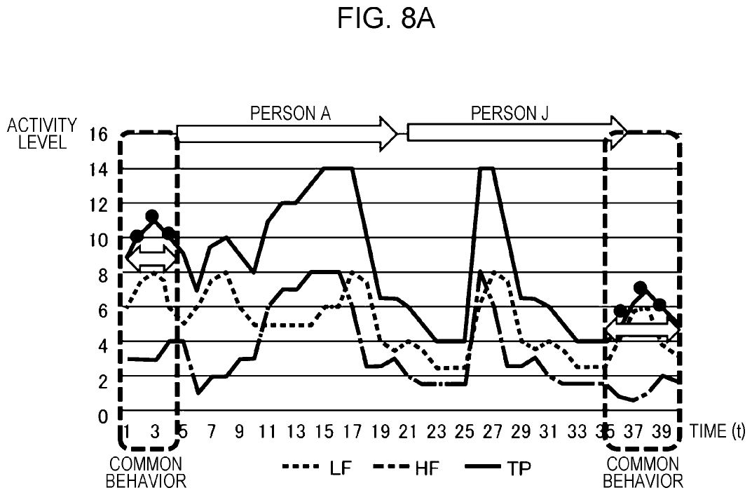

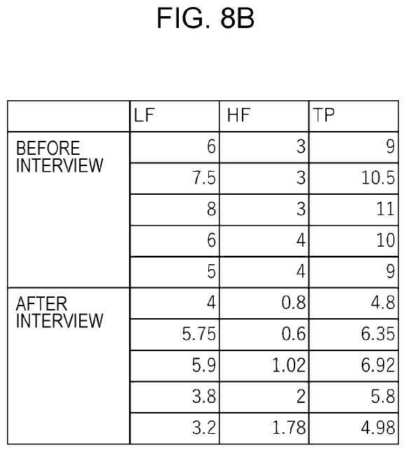

In step 100 of FIG. 7, the acquisition unit 30 acquires the biological information of the user stored in the storage unit 14, as illustrated in FIG. 8A as one example.

In step 102, with respect to the biological information of the user acquired in step 100, the setting unit 32 specifies segments of common behavior performed before and after the interviews as illustrated in FIG. 8A as one example. Additionally, the setting unit 32 creates the biological data table illustrated in FIG. 8B as one example from the biological information corresponding to the specified segment of the common behavior.

FIG. 8A is a graph illustrating one example of biological information of the user according to the present exemplary embodiment.

In FIG. 8A, the vertical axis indicates activity level while the horizontal axis indicates time. The dashed line indicates the LF component, the chain line indicates the HF component, and the solid line indicates the TP. Note that although the example illustrated in FIG. 8A illustrates only the biological information with respect to each of the person A and the person J, in actuality, biological information is acquired with respect to each of the ten persons from the person A to the person J.

FIG. 8B is a diagram illustrating one example of the biological data table according to the present exemplary embodiment.

In the biological data table illustrated in FIG. 8B, five sets of biological data (LF component, HF component, and the TP) are registered as the biological information of the user corresponding to the common behavior for each of both before the interviews and after the interviews.

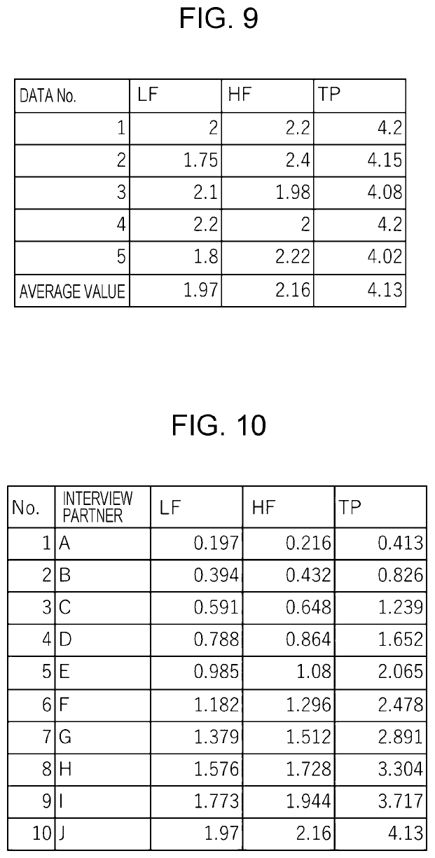

In step 104, the setting unit 32 computes the differences between the biological data before the interviews and the biological data after the interviews from the biological data table illustrated in FIG. 8B as one example, additionally computes the average value of the differences, and creates a difference data table illustrated in FIG. 9 as one example.

FIG. 9 is a diagram illustrating one example of the difference data table according to the present exemplary embodiment.

In the difference data table illustrated in FIG. 9, the differences between before and after the interviews are registered. For example, in the case of the biological data with the data number 1, a difference in the LF component of (6−4)=2, a difference in the HF component of (3−0.8)=2.2, and a difference in the TP of (9−4.8)=4.2 are registered. Biological data for the other data numbers 2 to 5 are also registered similarly. Additionally, 1.97, 2.16, and 4.13 are registered as the average values of the differences for the LF component, the HF component, and the TP, respectively.

In step 106, the setting unit 32 uses the average values of the differences obtained from the difference data table illustrated in FIG. 9 as one example to compute weight values for each interview partner and create a weight value table illustrated in FIG. 10 as one example. Herein, the weight values are obtained by dividing the average value of the difference between the TP in the common behavior before the interviews and the TP in the common behavior after the interviews by the number of interview partners, and multiplying by a value expressing the order. In other words, the weight values are computed using Formula (1) indicated below. Note that W1 is the weight value, Av is the average value of the difference, N is the number of interview partners, and T is a value expressing the order.

W1=(Av/N)×T (1)

FIG. 10 is a diagram illustrating one example of the weight value table according to the present exemplary embodiment.

In the weight value table illustrated in FIG. 10, weight values are registered for every interview partner. For example, in the case of the person A, given that N=10 and T=1, W1=(1.97/10)×1=0.197 is registered as the LF component, W1=(2.16/10)×1=0.216 is registered as the HF component, and W1=(4.13/10)×1=0.413 is registered as the TP. Similarly, in the case of the person J, given that N=10 and T=10, W1=(1.97/10)×10=1.97 is registered as the LF component, W1=(2.16/10)×10=2.16 is registered as the HF component, and W1=(4.13/10)×10=4.13 is registered as the TP. Weight values are registered similarly for the other interview partners, namely the persons B to I. In this way, the weight values are set to be larger for later persons compared to earlier persons in the interview order.

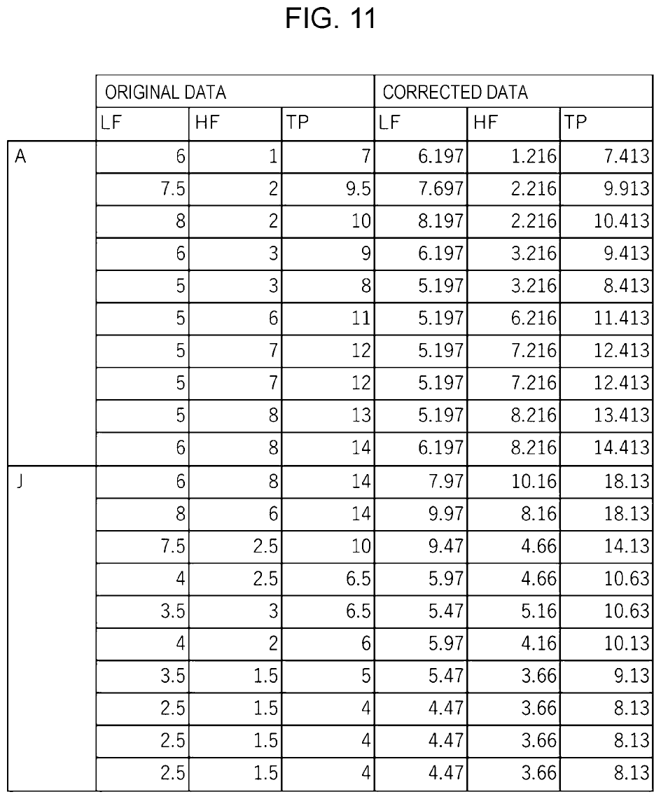

In step 108, the correction unit 34 corrects the biological information of the user on the basis of the weight values obtained from the weight value table illustrated in FIG. 10 as one example, creates a corrected data table illustrated in FIG. 11 as one example, and ends the series of processes by the impression measurement processing program 14A. At this point, the values of the corrected data are computed using Formula (2) indicated below. Note that Dc is the value of the corrected data, Do is the value of the original data, and W1 is the weight value.

Dc=Dc+W1 (2)

FIG. 11 is a diagram illustrating one example of the corrected data table according to the present exemplary embodiment.

In the corrected data table illustrated in FIG. 11, the values of the corrected data for every interview partner in the case of taking ten data points for each interview partner are registered. For example, in the case of the first set of biological data for the person A, for the LF component, given that Do=6 and that W1=0.197 for the LF component, Dc=6+0.197=6.197 is registered. Similarly, for the HF component, given that Do=1 and that W1=0.216 for the HF component, Dc=1+0.216=1.216 is registered, while for the TP, given that Do=7 and that W1=0.413 for the TP, Dc=7+0.413=7.413 is registered. Similarly, values of the corrected data are also registered for the second to tenth sets of biological data for the person A. Note that although the example illustrated in FIG. 11 illustrates only the corrected data for each of the person A and the person J, in actuality, the values of the corrected data for each of the ten persons from the person A to the person J are registered.

Note that the weight value W1 may be applied directly or a value obtained by rounding the weight value W1 may be applied to the correction coefficient set in the correction table 14B illustrated in FIG. 6A described above.

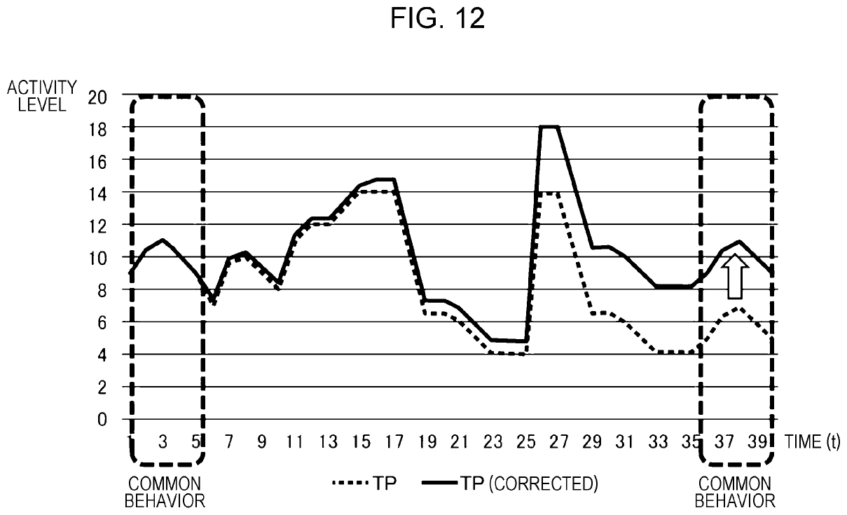

FIG. 12 is a graph illustrating one example of corrected biological information according to the present exemplary embodiment.

In FIG. 12, the vertical axis indicates activity level while the horizontal axis indicates time. The dashed line indicates the TP before correction, while the solid line indicates the corrected TP.

In the example illustrated in FIG. 12, the corrected TP that has been corrected using the corrected data table illustrated in FIG. 11 as one example is illustrated. In this way, the TP is corrected such that the activity level becomes larger.

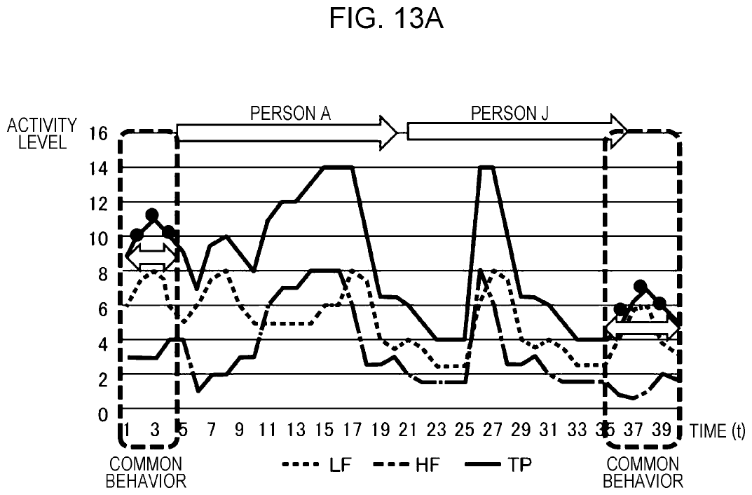

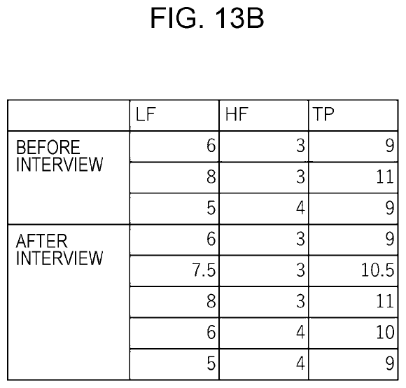

Next, FIGS. 13 to 16 will be referenced to describe the case of correcting the TP response time to become shorter.

First, the acquisition unit 30 acquires the biological information of the user stored in the storage unit 14, as illustrated in FIG. 13A as one example.

With respect to the biological information of the user acquired by the acquisition unit 30, the setting unit 32 specifies the segments of common behavior performed before and after the interviews as illustrated in FIG. 13A as one example. Additionally, the setting unit 32 creates the biological data table illustrated in FIG. 13B as one example from the biological information corresponding to the specified segment of the common behavior.

FIG. 13A is a graph illustrating another example of biological information of the user according to the present exemplary embodiment.

In FIG. 13A, the vertical axis indicates activity level while the horizontal axis indicates time. The dashed line indicates the LF component, the chain line indicates the HF component, and the solid line indicates the TP. Note that although the example illustrated in FIG. 13A illustrates only the biological information with respect to each of the person A and the person J, in actuality, biological information is acquired with respect to each of the ten persons from the person A to the person J.

FIG. 13B is a diagram illustrating another example of the biological data table according to the present exemplary embodiment.

In the biological data table illustrated in FIG. 13B, biological data (LF component, HF component, and the TP) equal to the number of data points are registered as the biological information of the user corresponding to the common behavior for each of both before the interviews and after the interviews. In the example illustrated in FIG. 13B, there are three data points before the interviews and five data points after the interviews.

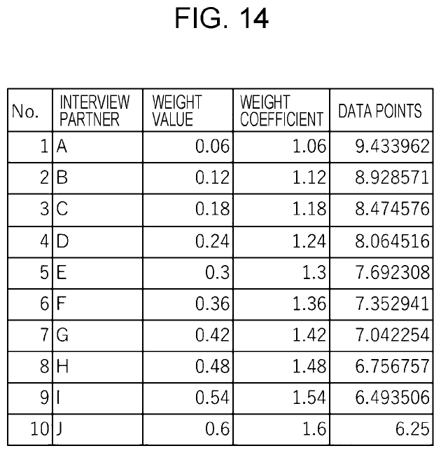

The 32 computes a compression ratio of before and after the interviews from the biological data table illustrated in FIG. 13B as one example. The compression ratio is computed as the number of data points before the interviews divided by the number of data points after the interviews. In the example illustrated in FIG. 13B, the compression ratio is computed to be 3/5=0.6. Subsequently, on the basis of the above compression ratio, the setting unit 32 computes a weight value, a weight coefficient, and a number of data points after correction for every interview partner, and creates a weight value table illustrated in FIG. 14 as one example. Herein, the weight values are obtained by taking the compression ratio obtained by dividing the number of data points for the TP in the common behavior before the interviews by the number of data points for the TP in the common behavior after the interviews, dividing by the number of interview partners, and multiplying by a value expressing the order. In other words, the weight values are computed using Formula (3) indicated below. Note that W2 is the weight value, Cp is the compression ratio, N is the number of interview partners, and T is a value expressing the order.

W2=(Cp/N)×T (3)

Also, the weight coefficients are computed using Formula (4) indicated below, and the number of data points after correction are computed using Formula (5) indicated below. Note that Wc is the weight coefficient, B is a constant equal to 1 or greater (set to 1, for example), Mb is the number of data points before correction (set to 10, for example), and Ma is the number of data points after correction.

Wc=B+W2 (4)

Ma=Mb/Wc (5)

FIG. 14 is a diagram illustrating another example of the weight value table according to the present exemplary embodiment.

In the weight value table illustrated in FIG. 14, the weight value, the weight coefficient, and the number of data points after correction are registered for every interview partner. For example, in the case of the person A, given that Cp=0.6, N=10, T=1, B=1, and Mb=10, W2=(0.6/10)×1=0.06, Wc=1+0.06=1.06, and Ma=10/1.069.43 are registered. Similarly, in the case of the person J, given that Cp=0.6, N=10, T=10, B=1, and Mb=10, W2=(0.6/10)×1=0.06, Wc=1+0.6=1.6, and Ma=10/1.6=6.25 are registered. Weight values, weight coefficients, and numbers of data points after correction are registered similarly for the other interview partners, namely the persons B to I. In this way, the weight values and the weight coefficients are set to be larger for later persons compared to earlier persons in the interview order.

The correction unit 34 corrects the biological information of the user on the basis of the number of data points after correction obtained from the weight value table illustrated in FIG. 14 as one example. Specifically, the correction unit 34 performs a process of compressing the number of data points before correction according to the number of data points after correction, as illustrated in FIG. 15 as one example.

FIG. 15 is a diagram accompanying a description of data compression according to the present exemplary embodiment.

The diagram on the left in FIG. 15 illustrates the state of the biological data before correction, while the diagram on the right in FIG. 15 illustrates the state of the biological data after correction. For example, in the case of the person A, the number of data points is compressed from 10 before correction to 9 after correction. Similarly, in the case of the person J, the number of data points is compressed from 10 before correction to 6 after correction. The numbers of data points are compressed similarly for the other interview partners, namely the persons B to I. The method of compressing the number of data points may be, for example, culling any of the data, culling the data sequentially from the beginning, or culling every other data point.

Note that 1/Wc or a value obtained by rounding 1/Wc may be applied to the correction coefficient set in the correction table 14B illustrated in FIG. 6A described above.

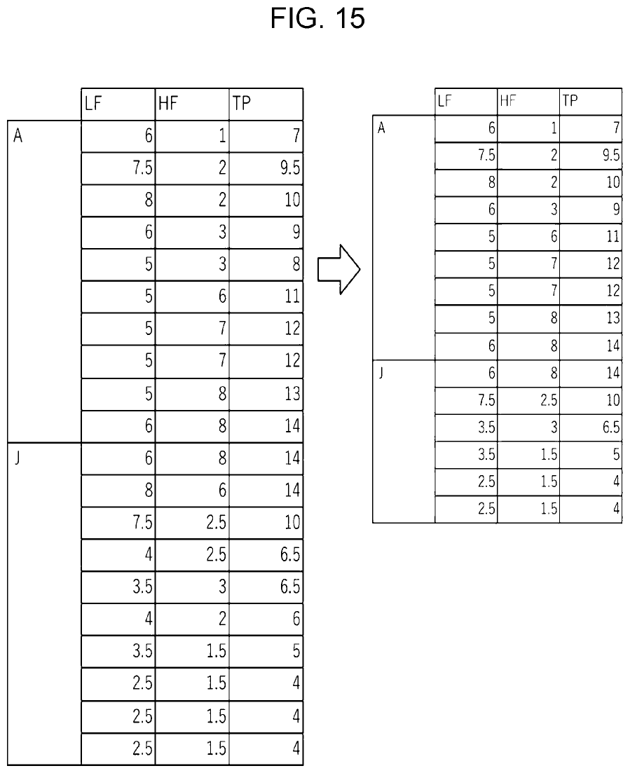

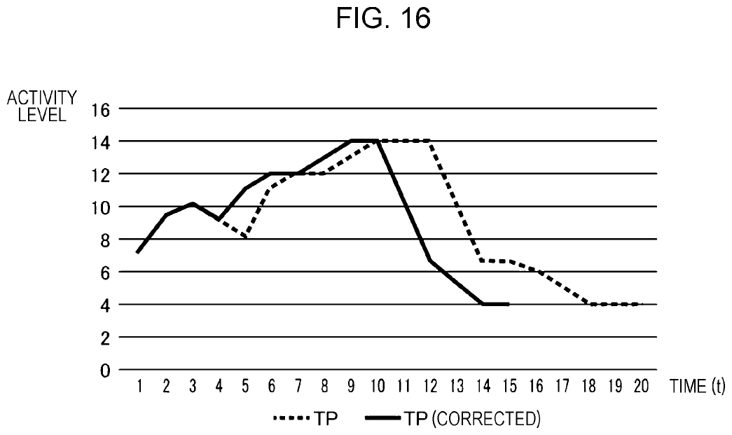

FIG. 16 is a graph illustrating another example of corrected biological information according to the present exemplary embodiment.

In FIG. 16, the vertical axis indicates activity level while the horizontal axis indicates time. The dashed line indicates the TP before correction, while the solid line indicates the corrected TP.

In the example illustrated in FIG. 16, the corrected TP that has been corrected using the weight value table illustrated in FIG. 14 as one example is illustrated. In this way, the TP is corrected such that the response time becomes shorter.

The above describes each of the case of correcting the biological information of the user such that the activity level becomes larger, and the case of correcting the biological information of the user such that the response time becomes shorter. The present exemplary embodiment is not limited to these corrections, and may also correct the biological information of the user such that the activity level becomes larger and the response time becomes shorter at the same time.

According to the present exemplary embodiment, when the user interviews multiple persons in order, the impression of each partner being influenced by the interview order is suppressed. For this reason, the impression of each partner is measured accurately irrespectively of the interview order.

### Second Exemplary Embodiment

The first exemplary embodiment above describes a configuration that corrects the biological information of the user according to the order of interview partners, but as a result of the correction, cases may occur in which the biological response to a partner earlier in the order and the biological response to a partner later in the order become substantially the same, as illustrated in FIG. 17 as one example. The present exemplary embodiment describes a configuration that ranks the impressions of the interview partners using a slope score, an interview order score, and the like for every interview partner.

FIG. 17 is a graph illustrating one example of biological information according to the second exemplary embodiment.

In FIG. 17, the vertical axis indicates activity level while the horizontal axis indicates time. The biological information illustrated in FIG. 17 is the corrected TP.

In the example illustrated in FIG. 17, as a result of the correction in the first exemplary embodiment above, the biological response with respect to an early partner (person A) in the interview order and the biological response with respect to a later partner (person J) have become substantially the same. However, if the user's fatigue and the like are considered, even if the biological responses are substantially the same, the impression of the later partner (person J) in the interview order is better (more favorable) in some cases.

For this reason, by having the CPU 12A of an impression measurement device 11 according to the present exemplary embodiment load the impression measurement processing program 14A stored in the storage unit 14 into the RAM 12C and execute the impression measurement processing program 14A, the CPU 12A functions as each unit illustrated in FIG. 18.

FIG. 18 is a block diagram illustrating one example of a functional configuration of the impression measurement device 11 according to the second exemplary embodiment.

As illustrated in FIG. 18, the CPU 12A of the impression measurement device 11 according to the present exemplary embodiment functions as an acquisition unit 30, a setting unit 32, a correction unit 34, a derivation unit 36, and a display control unit 38. Note that the display control unit 38 is one example of a control unit. Also, in the present exemplary embodiment, since the acquisition unit 30, the setting unit 32, and the correction unit 34 are similar to the impression measurement device 10 according to the first exemplary embodiment above, a repeated description is omitted here.

The derivation unit 36 according to the present exemplary embodiment assigns a slope score and an interview order score to every interview partner on the basis of the biological information corrected by the correction unit 34, and derives a total score for every interview partner from the slope score and the interview order score. Note that the slope score refers to a score with respect to the slope of the graph indicated the biological information. As one example, the slope score is assigned a larger value to the extent that the slope of the graph is larger in the positive direction. Also, the interview order score is a score with respect to the order of interviews. As one example, the interview order score is assigned a larger value for partners later in the order of interviews.

Herein, the derivation unit 36 may additionally use the behavior information obtained from the behavior of the user and assign a behavior score, which is a score with respect to the behavior information, to every interview partner. Note that the behavior information is acquired by the behavior information detection unit 54, as described earlier. As one example, the behavior of the user referred to herein includes hand movements, nodding, leaning forward, the degree of upturning at the corners of the mouth, smiling, and the like. These behaviors are acquired by analyzing an image obtained by photographing or recording video of the user. Specifically, in the case in which the user's hands make large movements (for example, in the case where the number of hand movements is equal to or greater than a predetermined number) or the user is leaning forward (for example, in the case where the lean-forward angle is equal to or greater than a predetermined angle), the probability that the user has a good impression of the partner is considered to be high, and therefore a large value is assigned. Also, in the case in which the number of nods by the user is high (for example, in the case where the number of nods is equal to or greater than a predetermined number), the probability that the user does not have a very good impression of the partner is considered to be high, and therefore a small value is assigned. Also, in the case in which the degree of upturning at the corners of the user's mouth (for example, in the case in which the angle of the corners of the mouth is equal to or greater than a predetermined angle) or the user is smiling, the probability that the user has a good impression of the partner is considered to be high, and therefore a large value is assigned. In this case, the derivation unit 36 derives the total score for every interview partner from the slope score, the interview order score, and the behavior score.

The display control unit 38 according to the present exemplary embodiment controls the display of a rank table that ranks each of the interview partners on the basis of the total score for every interview partner derived by the derivation unit 36. The tank table may be displayed on the display unit 16 or displayed on a portable terminal or the like used by the user.

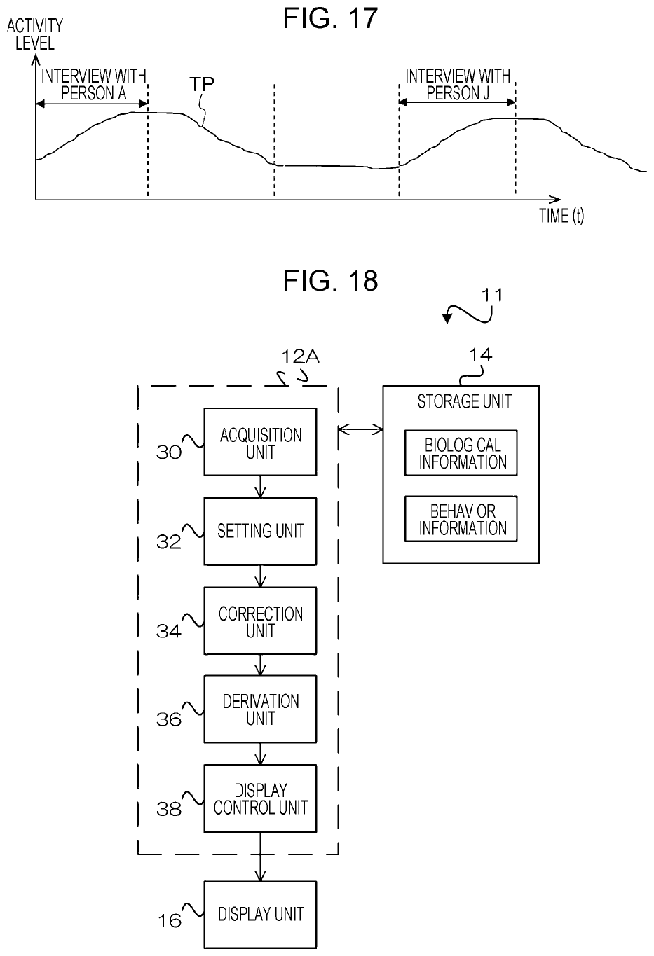

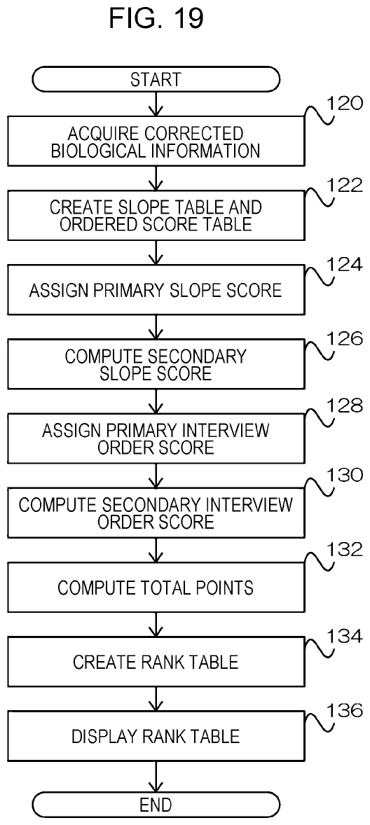

Next, FIGS. 19 to 27 will be referenced to describe the action of the impression measurement device 11 according to the second exemplary embodiment. Herein, a case in which a positive (+) slope and a negative (−) slope appear alternately in a graph indicating the corrected biological information will be described.

FIG. 19 is a flowchart illustrating one example of the flow of a process by the impression measurement processing program 14A according to the second exemplary embodiment.

First, if the impression measurement device 11 is instructed by a person in charge of measurement to launch the impression measurement processing program 14A, each of the following steps is executed. Note that the corrected biological information and the behavior information described above are stored in the storage unit 14, but in the present exemplary embodiment, it is assumed that a behavior score is not assigned.

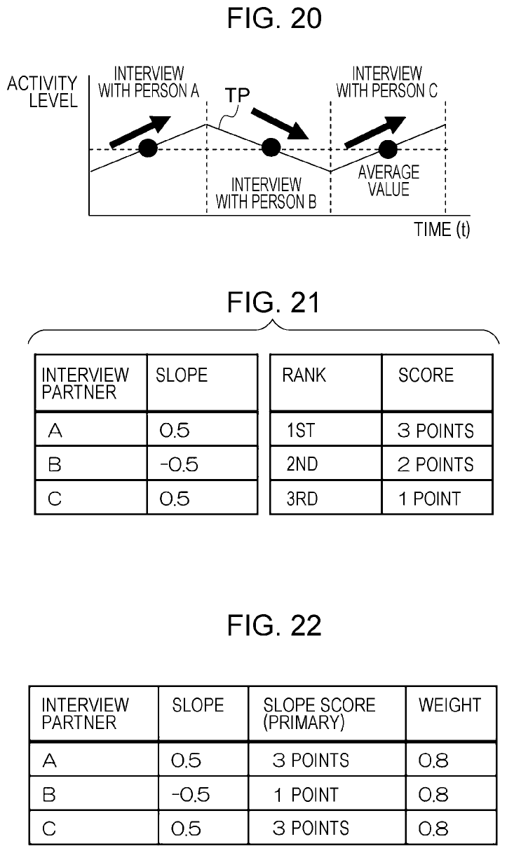

In step 120 of FIG. 19, the acquisition unit 30 acquires the corrected biological information stored in the storage unit 14, as illustrated in FIG. 20 as one example.

FIG. 20 is a graph illustrating one example of corrected biological information according to the present exemplary embodiment.

In FIG. 20, the vertical axis indicates activity level while the horizontal axis indicates time. The biological information illustrated in FIG. 20 is the TP. Note that although the example illustrated in FIG. 20 illustrates only the biological information with respect to each of the persons A to C for the sake of simplicity, in actuality, biological information is acquired with respect to each of the ten persons from the person A to the person J.

In step 122, the derivation unit 36 creates a slope table and a rank score table, as illustrated in FIG. 21 as one example.

FIG. 21 is a diagram illustrating one example of the slope table and the rank score table according to the present exemplary embodiment.

The diagram on the left in FIG. 21 illustrates the slope table of the graph indicating the biological information illustrated in FIG. 20, while the diagram on the right in FIG. 21 illustrates the score table with respect to the ranks of the slopes in the graph.

In the slope table illustrated in the diagram on the left in FIG. 21, 0.5 is registered as the slope of the graph for the person A illustrated in FIG. 20, −0.5 is registered as the slope of the graph for the person B, and 0.5 is registered as the slope of the graph for the person C. Also, in the rank score table illustrated in the diagram on the right in FIG. 21, a higher rank (herein, 1st is the highest rank) and a larger score are assigned to the extent that the slope of the graph is larger in the positive direction.

In step 124, the derivation unit 36 assigns a primary slope score, as illustrated in FIG. 22 as one example.

FIG. 22 is a diagram illustrating one example of the primary slope score according to the present exemplary embodiment.

The primary slope score illustrated in FIG. 22 is assigned on the basis of the slope table and the rank score table illustrated in FIG. 21. For example, in the case of the person A, since the slope of 0.5 is ranked 1st, 3 is assigned as the primary slope score. Similarly, in the case of the person B, since the slope of −0.5 is ranked 3rd, 1 is assigned as the primary slope score. In the case of the person C, since the slope of 0.5 is tied for 1st, 3 is assigned as the primary slope score. Note that a weight for the slope (slope weight) is assumed to be 0.8 for example.

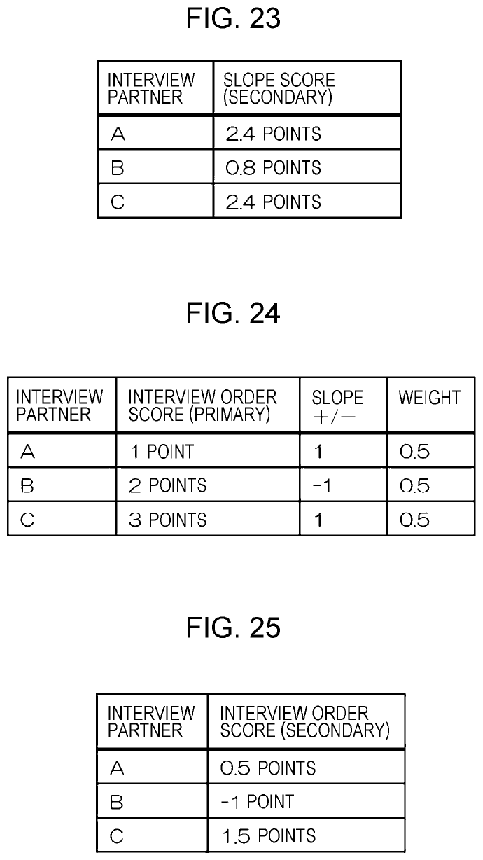

In step 126, the derivation unit 36 computes a secondary slope score, as illustrated in FIG. 23 as one example.

FIG. 23 is a diagram illustrating one example of the secondary slope score according to the present exemplary embodiment.

The secondary slope score illustrated in FIG. 23 is computed according to Formula (6) indicated below as one example. Note that S1 is the primary slope score, S2 is the secondary slope score, and Wk is the slope weight.

S2=S1×Wk (6)

As illustrated in FIG. 23, for example, in the case of the person A, a secondary slope score of S2=3×0.8=2.4 points is computed. Similarly, in the case of the person B, a secondary slope score of S2=1×0.8=0.8 points is computed, and in the case of the person C, a secondary slope score of S2=3×0.8=2.4 points is computed.

In step 128, the derivation unit 36 assigns a primary interview order score, as illustrated in FIG. 24 as one example.

FIG. 24 is a diagram illustrating one example of the primary interview order score according to the present exemplary embodiment.

The primary interview order score illustrated in FIG. 24 is assigned a larger value for partners later in the order of interviews.

For example, since the person A is first in the order of interviews, 1 is assigned as the primary interview order score. Similarly, since the person B is second in the order of interviews, 2 is assigned as the primary interview order score. Since the person C is third in the order of interviews, 3 is assigned as the primary interview order score. Note that a weight for the order of interviews (interview order weight) is assumed to be 0.5 for example.

At this point, it is desirable for the interview order score to reflect whether the slope of the graph is positive or negative. For example, a negative slope indicates that the favorableness is falling, while a positive slope indicates that the favorableness is rising. For this reason, by reflecting not only the order of interviews but also the sign of the slope of the graph, a more appropriate score is computed.

In step 130, the derivation unit 36 computes a secondary interview order score, as illustrated in FIG. 25 as one example.

FIG. 25 is a diagram illustrating one example of the secondary interview order score according to the present exemplary embodiment.

The secondary interview order score illustrated in FIG. 25 is computed according to Formula (7) indicated below as one example. Note that S11 is the primary interview order score, S12 is the secondary interview order score, P is the sign of the slope, and Wm is the interview order weight.

S12=S11×P×Wm (7)

As illustrated in FIG. 25, for example, in the case of the person A, a secondary interview order score of S12=1×1×0.5=0.5 points is computed. Similarly, in the case of the person B, a secondary interview order score of S12=2×(−1)×0.5=−1 point is computed, and in the case of the person C, a secondary interview order score of S12=3×1×0.5=1.5 points is computed.

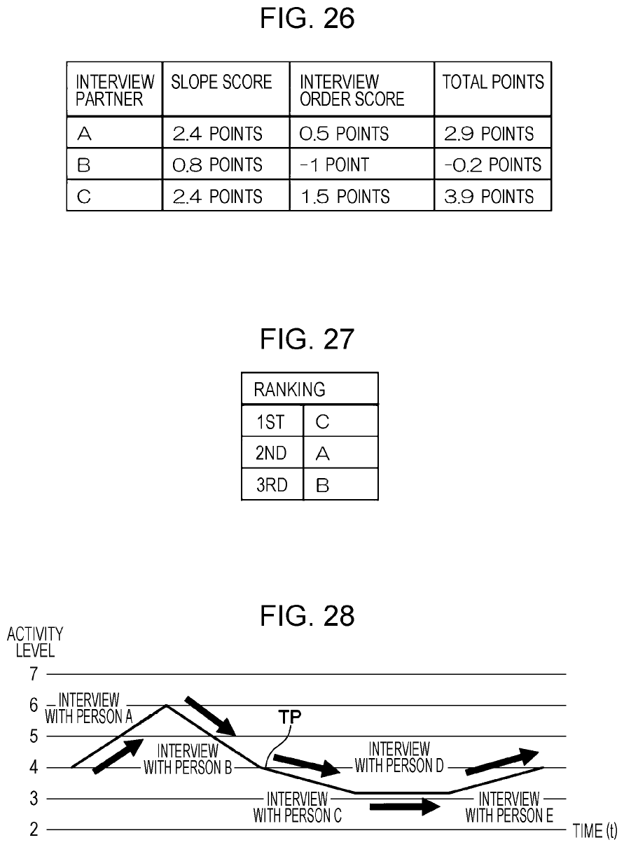

In step 132, the derivation unit 36 computes a total score (total points) for every interview partner from the secondary slope score computed in step 126 and the secondary interview order score computed in step 130, as illustrated in FIG. 26 as one example.

FIG. 26 is a diagram illustrating one example of the total score according to the present exemplary embodiment.

The total score illustrated in FIG. 26 is computed as the total of the secondary slope score and the secondary interview order score.

As illustrated in FIG. 26, for example, in the case of the person A, since the secondary slope score is 2.4 points and the secondary interview order score is 0.5 points, the total score is computed to be 2.9 points. Similarly, in the case of the person B, since the secondary slope score is 0.8 points and the secondary interview order score is −1 point, the total score is computed to be −0.2 points, and in the case of the person C, since the secondary slope score is 2.4 points and the secondary interview order score is 1.5 points, the total score is computed to be 3.9 points.

In step 134, the derivation unit 36 creates a rank table that ranks each of the interview partners from the total score computed in step 132, as illustrated in FIG. 27 as one example.

FIG. 27 is a diagram illustrating one example of the rank table according to the present exemplary embodiment.

The rank table illustrated in FIG. 27 ranks the interview partners in order of the highest total score illustrated in FIG. 26. Herein, the ranking becomes person C, person A, person B. In other words, it is determined that the person C is the partner who made the best (most favorable) impression on the user.

In step 136, the display control unit 38 causes the rank table created in step 134 to be displayed on a portable terminal or the like used by the user as one example, and ends the series of processes by the impression measurement processing program 14A.

Next, FIGS. 28 to 35 will be referenced to describe a case in which a negative slope appears for two or more persons consecutively in the graph indicating the corrected biological information.

First, the acquisition unit 30 acquires the corrected biological information stored in the storage unit 14, as illustrated in FIG. 28 as one example.

FIG. 28 is a graph illustrating another example of corrected biological information according to the present exemplary embodiment.

In FIG. 28, the vertical axis indicates activity level while the horizontal axis indicates time. The biological information illustrated in FIG. 28 is the TP. Note that although the example illustrated in FIG. 28 illustrates only the biological information with respect to each of the persons A to E for the sake of simplicity, in actuality, biological information is acquired with respect to each of the ten persons from the person A to the person J.

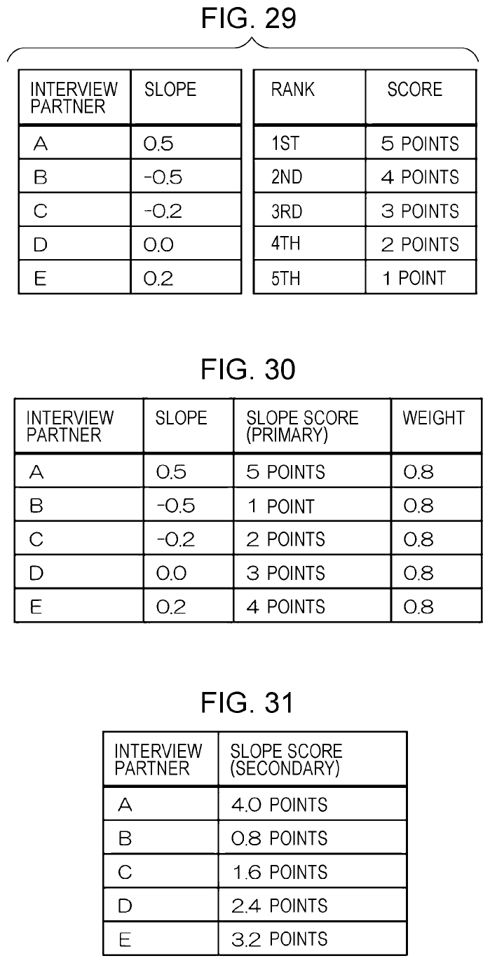

The derivation unit 36 creates a slope table and a rank score table, as illustrated in FIG. 29 as one example.

FIG. 29 is a diagram illustrating another example of the slope table and the rank score table according to the present exemplary embodiment.

The diagram on the left in FIG. 29 illustrates the slope table of the graph indicating the biological information illustrated in FIG. 28, while the diagram on the right in FIG. 29 illustrates the score table with respect to the ranks of the slopes in the graph.

In the slope table illustrated in the diagram on the left in FIG. 29, 0.5 is registered as the slope of the graph for the person A illustrated in FIG. 28, −0.5 is registered as the slope of the graph for the person B, −0.2 is registered as the slope of the graph for the person C, 0.0 is registered as the slope of the graph for the person D, and 0.2 is registered as the slope of the graph for the person E. Also, in the rank score table illustrated in the diagram on the right in FIG. 29, a higher rank (herein, 1st is the highest rank) and a larger score are assigned to the extent that the slope of the graph is larger in the positive direction.

The derivation unit 36 assigns a primary slope score, as illustrated in FIG. 30 as one example.

FIG. 30 is a diagram illustrating another example of the primary slope score according to the present exemplary embodiment.

The primary slope score illustrated in FIG. 30 is assigned on the basis of the slope table and the rank score table illustrated in FIG. 29.

For example, in the case of the person A, since the slope of 0.5 is ranked 1st, 5 is assigned as the primary slope score. Similarly, in the case of the person B, since the slope of −0.5 is ranked 5th, 1 is assigned as the primary slope score. In the case of the person C, since the slope of −0.2 is ranked 4th, 2 is assigned as the primary slope score. In the case of the person D, since the slope of 0.0 is ranked 3rd, 3 is assigned as the primary slope score. In the case of the person E, since the slope of 0.2 is ranked 2nd, 4 is assigned as the primary slope score. Note that a weight for the slope (slope weight) is assumed to be 0.8 for example.

The derivation unit 36 computes a secondary slope score, as illustrated in FIG. 31 as one example.

FIG. 31 is a diagram illustrating another example of the secondary slope score according to the present exemplary embodiment.

The secondary slope score illustrated in FIG. 31 is computed according to Formula (6) described above as one example.

In other words, as illustrated in FIG. 31, for example, in the case of the person A, a secondary slope score of S2=5×0.8=4.0 points is computed. Similarly, in the case of the person B, a secondary slope score of S2=1×0.8=0.8 points is computed, and in the case of the person C, a secondary slope score of S2=2×0.8=1.6 points is computed. Also, in the case of the person D, a secondary slope score of S2=3×0.8=2.4 points is computed, and in the case of the person E, a secondary slope score of S2=4×0.8=3.2 points is computed.

The derivation unit 36 assigns a primary interview order score, as illustrated in FIG. 30 as one example.

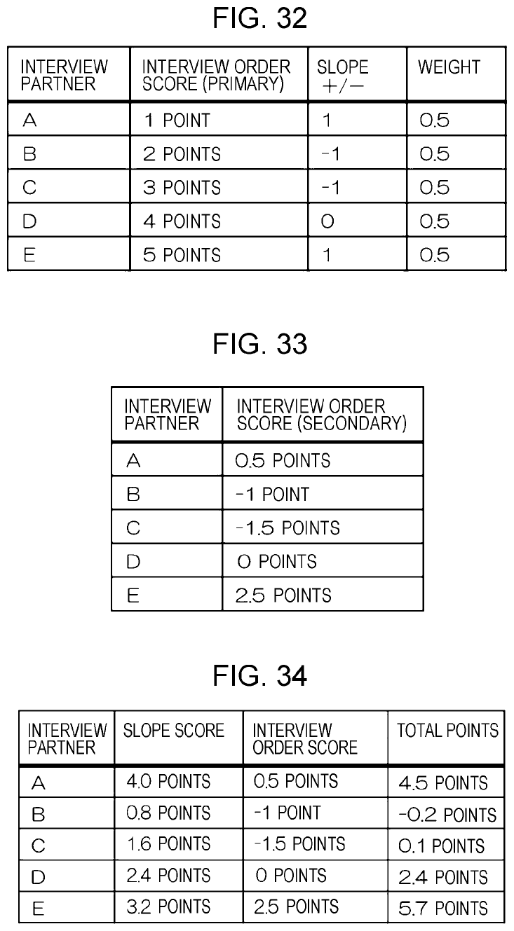

FIG. 32 is a diagram illustrating another example of the primary interview order score according to the present exemplary embodiment.

The primary interview order score illustrated in FIG. 32 is assigned a larger value for partners later in the order of interviews.

For example, since the person A is first in the order of interviews, 1 is assigned as the primary interview order score. Similarly, since the person B is second in the order of interviews, 2 is assigned as the primary interview order score. Since the person C is third in the order of interviews, 3 is assigned as the primary interview order score. Since the person D is fourth in the order of interviews, 4 is assigned as the primary interview order score. Since the person E is fifth in the order of interviews, 5 is assigned as the primary interview order score. Note that a weight for the order of interviews (interview order weight) is assumed to be 0.5 for example.

At this point, similarly to the example illustrated in FIG. 24 described above, it is desirable for the interview order score to reflect whether the slope of the graph is positive (+) or negative (−).

The derivation unit 36 assigns a secondary interview order score, as illustrated in FIG. 33 as one example.

FIG. 33 is a diagram illustrating another example of the secondary interview order score according to the present exemplary embodiment.

The secondary interview order score illustrated in FIG. 33 is computed according to Formula (7) described above as one example.

In other words, as illustrated in FIG. 33, for example, in the case of the person A, a secondary interview order score of S12=1×1×0.5=0.5 points is computed. Similarly, in the case of the person B, a secondary interview order score of S12=2×(−1)×0.5=−1 point is computed, and in the case of the person C, a secondary interview order score of S12=3×(−1)×0.5=−1.5 points is computed. In the case of the person D, a secondary interview order score of S12=4×(0)×0.5=0 points is computed, and in the case of the person E, a secondary interview order score of S12=5×1×0.5=2.5 points is computed.

The derivation unit 36 computes a total score (total points) for every interview partner from the secondary slope score computed in FIG. 31 and the secondary interview order score computed in FIG. 33 described above, as illustrated in FIG. 34 as one example.

FIG. 34 is a diagram illustrating another example of the total score according to the present exemplary embodiment.

The total score illustrated in FIG. 34 is computed as the total of the secondary slope score and the secondary interview order score.

As illustrated in FIG. 34, for example, in the case of the person A, since the secondary slope score is 4.0 points and the secondary interview order score is 0.5 points, the total score is computed to be 4.5 points. Similarly, in the case of the person B, since the secondary slope score is 0.8 points and the secondary interview order score is −1 point, the total score is computed to be −0.2 points, and in the case of the person C, since the secondary slope score is 1.6 points and the secondary interview order score is −1.5 points, the total score is computed to be 0.1 points. In the case of the person D, since the secondary slope score is 2.4 points and the secondary interview order score is 0 points, the total score is computed to be 2.4 points, and in the case of the person E, since the secondary slope score is 3.2 points and the secondary interview order score is 2.5 points, the total score is computed to be 5.7 points.

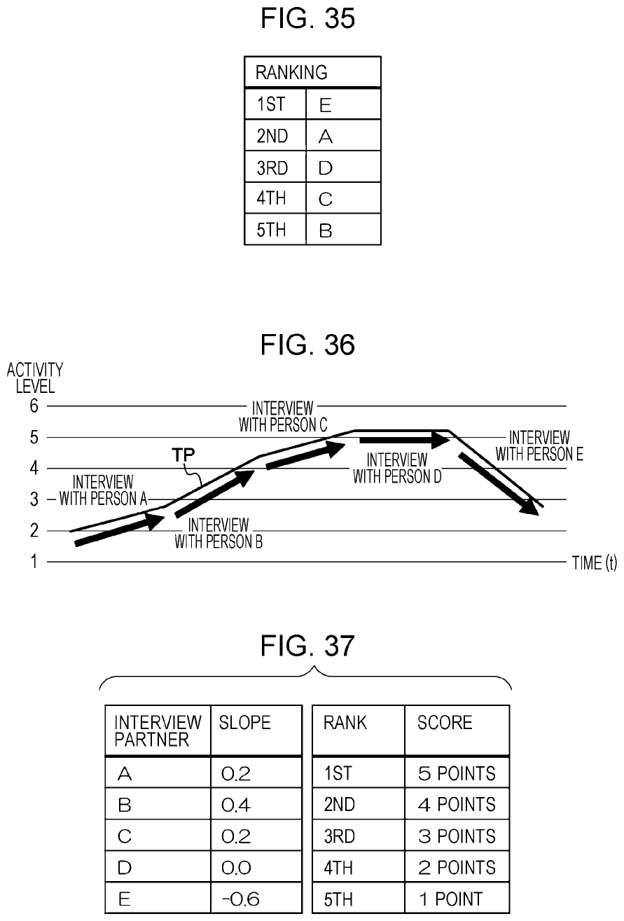

The derivation unit 36 creates a rank table that ranks each of the interview partners from the total score computed in FIG. 34 described above, as illustrated in FIG. 35 as one example.

FIG. 35 is a diagram illustrating another example of the rank table according to the present exemplary embodiment.

The rank table illustrated in FIG. 35 ranks the interview partners in order of the highest total score illustrated in FIG. 34. Herein, the ranking becomes person E, person A, person D, person C, person B. In other words, it is determined that the person E is the partner who made the best (most favorable) impression on the user.

The display control unit 38 causes the rank table created in FIG. 35 described above to be displayed on a portable terminal or the like used by the user as one example.

Next, FIGS. 36 to 43 will be referenced to describe a case in which a positive slope appears for two or more persons consecutively in the graph indicating the corrected biological information.

First, the acquisition unit 30 acquires the corrected biological information stored in the storage unit 14, as illustrated in FIG. 36 as one example.

FIG. 36 is a graph illustrating another example of corrected biological information according to the present exemplary embodiment.

In FIG. 36, the vertical axis indicates activity level while the horizontal axis indicates time. The biological information illustrated in FIG. 36 is the TP. Note that although the example illustrated in FIG. 36 illustrates only the biological information with respect to each of the persons A to E for the sake of simplicity, in actuality, biological information is acquired with respect to each of the ten persons from the person A to the person J.

The derivation unit 36 creates a slope table and a rank score table, as illustrated in FIG. 37 as one example.

FIG. 37 is a diagram illustrating another example of the slope table and the rank score table according to the present exemplary embodiment.

The diagram on the left in FIG. 37 illustrates the slope table of the graph indicating the biological information illustrated in FIG. 36, while the diagram on the right in FIG. 37 illustrates the score table with respect to the ranks of the slopes in the graph.

In the slope table illustrated in the diagram on the left in FIG. 37, 0.2 is registered as the slope of the graph for the person A illustrated in FIG. 36, 0.4 is registered as the slope of the graph for the person B, 0.2 is registered as the slope of the graph for the person C, 0.0 is registered as the slope of the graph for the person D, and −0.6 is registered as the slope of the graph for the person E. Also, in the rank score table illustrated in the diagram on the right in FIG. 37, a higher rank (herein, 1st is the highest rank) and a larger score are assigned to the extent that the slope of the graph is larger in the positive direction.

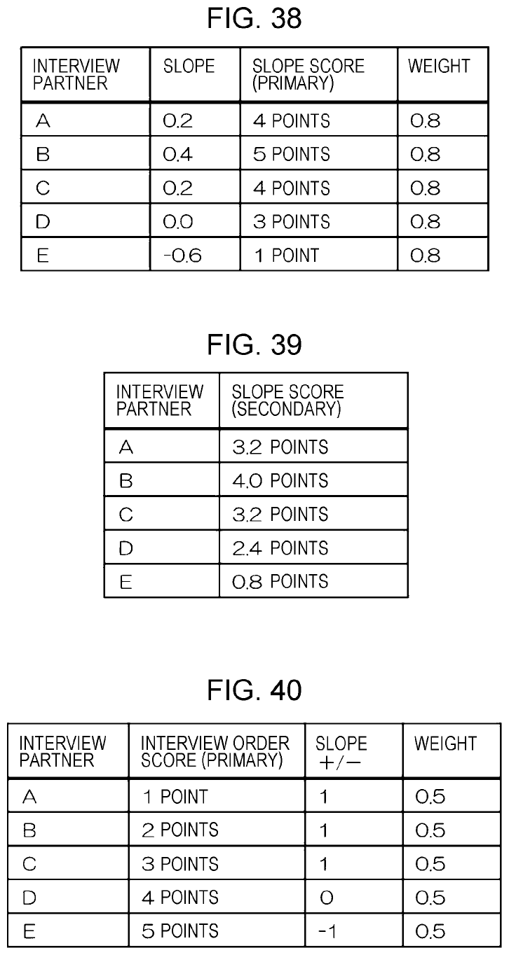

The derivation unit 36 assigns a primary slope score, as illustrated in FIG. 38 as one example.

FIG. 38 is a diagram illustrating another example of the primary slope score according to the present exemplary embodiment.

The primary slope score illustrated in FIG. 38 is assigned on the basis of the slope table and the rank score table illustrated in FIG. 37.

For example, in the case of the person A, since the slope of 0.2 is ranked 2nd, 4 is assigned as the primary slope score. Similarly, in the case of the person B, since the slope of 0.4 is ranked 1st, 5 is assigned as the primary slope score. In the case of the person C, since the slope of 0.2 is tied for 2nd, 4 is assigned as the primary slope score. In the case of the person D, since the slope of 0.0 is ranked 3rd, 3 is assigned as the primary slope score. In the case of the person E, since the slope of −0.6 is ranked 5th, 1 is assigned as the primary slope score. Note that a weight for the slope (slope weight) is assumed to be 0.8 for example.

The derivation unit 36 computes a secondary slope score, as illustrated in FIG. 39 as one example.

FIG. 39 is a diagram illustrating another example of the secondary slope score according to the present exemplary embodiment.

The secondary slope score illustrated in FIG. 39 is computed according to Formula (6) described above as one example.

In other words, as illustrated in FIG. 39, for example, in the case of the person A, a secondary slope score of S2=4×0.8=3.2 points is computed. Similarly, in the case of the person B, a secondary slope score of S2=5×0.8=4.0 points is computed, and in the case of the person C, a secondary slope score of S2=4×0.8=3.2 points is computed. Also, in the case of the person D, a secondary slope score of S2=3×0.8=2.4 points is computed, and in the case of the person E, a secondary slope score of S2=1×0.8=0.8 points is computed.

The derivation unit 36 assigns a primary interview order score, as illustrated in FIG. 40 as one example.

FIG. 40 is a diagram illustrating another example of the primary interview order score according to the present exemplary embodiment.

The primary interview order score illustrated in FIG. 40 is assigned a larger value for partners later in the order of interviews.

For example, since the person A is first in the order of interviews, 1 is assigned as the primary interview order score. Similarly, since the person B is second in the order of interviews, 2 is assigned as the primary interview order score. Since the person C is third in the order of interviews, 3 is assigned as the primary interview order score. Since the person D is fourth in the order of interviews, 4 is assigned as the primary interview order score. Since the person E is fifth in the order of interviews, 5 is assigned as the primary interview order score. Note that a weight for the order of interviews (interview order weight) is assumed to be 0.5 for example.

At this point, similarly to the example illustrated in FIG. 24 described above, it is desirable for the interview order score to reflect whether the slope of the graph is positive (+) or negative (−).

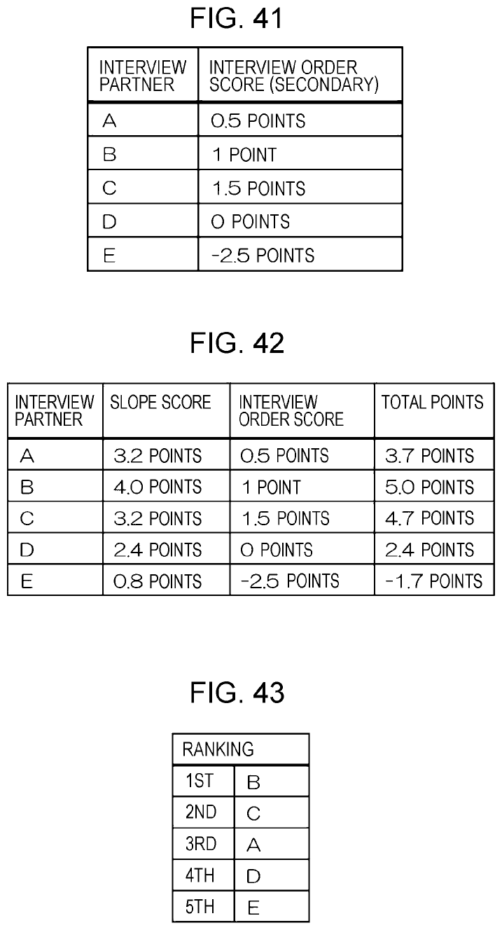

The derivation unit 36 assigns a secondary interview order score, as illustrated in FIG. 41 as one example.

FIG. 41 is a diagram illustrating another example of the secondary interview order score according to the present exemplary embodiment.

The secondary interview order score illustrated in FIG. 41 is computed according to Formula (7) described above as one example.

In other words, as illustrated in FIG. 41, for example, in the case of the person A, a secondary interview order score of S12=1×1×0.5=0.5 points is computed. Similarly, in the case of the person B, a secondary interview order score of S12=2×1×0.5=1 point is computed, and in the case of the person C, a secondary interview order score of 512=3×1×0.5=1.5 points is computed. In the case of the person D, a secondary interview order score of S12=4×(0)×0.5=0 points is computed, and in the case of the person E, a secondary interview order score of 512=5×(−1)×0.5=−2.5 points is computed.

The derivation unit 36 computes a total score (total points) for every interview partner from the secondary slope score computed in FIG. 41 and the secondary interview order score computed in FIG. 39 described above, as illustrated in FIG. 42 as one example.

FIG. 42 is a diagram illustrating another example of the total score according to the present exemplary embodiment.

The total score illustrated in FIG. 42 is computed as the total of the secondary slope score and the secondary interview order score.

As illustrated in FIG. 42, for example, in the case of the person A, since the secondary slope score is 3.2 points and the secondary interview order score is 0.5 points, the total score is computed to be 3.7 points. Similarly, in the case of the person B, since the secondary slope score is 4.0 points and the secondary interview order score is 1 point, the total score is computed to be 5.0 points, and in the case of the person C, since the secondary slope score is 3.2 points and the secondary interview order score is 1.5 points, the total score is computed to be 4.7 points. In the case of the person D, since the secondary slope score is 2.4 points and the secondary interview order score is 0 points, the total score is computed to be 2.4 points, and in the case of the person E, since the secondary slope score is 0.8 points and the secondary interview order score is −2.5 points, the total score is computed to be −1.7 points.

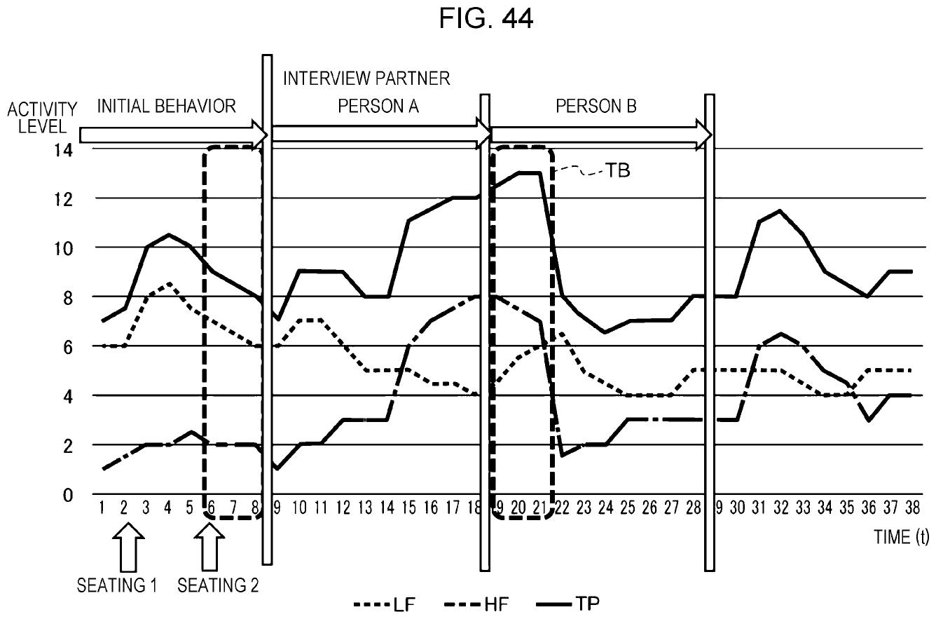

The derivation unit 36 creates a rank table that ranks each of the interview partners from the total score computed in FIG. 42 described above, as illustrated in FIG. 43 as one example.

FIG. 43 is a diagram illustrating another example of the rank table according to the present exemplary embodiment.

The rank table illustrated in FIG. 43 ranks the interview partners in order of the highest total score illustrated in FIG. 42. Herein, the ranking becomes person B, person C, person A, person D, person E. In other words, it is determined that the person B is the partner who made the best (most favorable) impression on the user.

The display control unit 38 causes the rank table created in FIG. 43 described above to be displayed on a portable terminal or the like used by the user as one example.

According to the present exemplary embodiment, as a result of the correction of the biological information, the slope score, the interview order score, and the like are used to rank the impressions of the interview partners, even in cases where the biological response to a partner earlier in the order and the biological response to a partner later in the order become substantially the same. For this reason, the impressions of the interview partners are grasped appropriately, irrespective of the order of interviews.

Next, FIG. 44 will be referenced to describe a configuration in which a correction target segment is removed from the biological information.

FIG. 44 is a graph illustrating another example of biological information before correction according to the present exemplary embodiment.

In FIG. 44, the vertical axis indicates activity level while the horizontal axis indicates time. The dashed line indicates the LF component, the chain line indicates the HF component, and the solid line indicates the TP. Note that although the example illustrated in FIG. 44 illustrates only the biological information with respect to each of the persons A and B for the sake of simplicity, in actuality, biological information is acquired with respect to each of the ten persons from the person A to the person J.

As illustrated in FIG. 44, biological information is acquired for an initial behavior that the user performs before the interviews (for example, seating 1→standing up→bowing→seating 2). Additionally, from the acquired biological information, the time after seating 2 until a state similar to seating 1 (for example, the TP value becomes the same value) is reached (hereinafter referred to as the “response extinction time”) is computed. Herein, the user takes a seat at time No. 6, and returns to the same TP value as seating 1 at time No. 8. In this case, the response extinction time is computed to be 3 minutes for example. This response extinction time is treated as a correction target time TB. Additionally, from the point in time when the interview with the person B starts after the interview with the person A, the segment from No. 19 to No. 21 is specified as one example of the correction target time TB. Additionally, the biological information in the segment of the specified correction target time TB is removed. Note that for simplicity, a predetermined time (such as 5 minutes, for example) may also be used as the response extinction time. In some cases, immediately after starting the interview with the person B, the user may still be influenced by the impression of the preceding interview partner, namely the person A. For this reason, by removing the biological information in the segment of the correction target time TB from the biological information with respect to the person B, the influence of the impression of the person A is reduced.

The above describes an example of an impression measurement device according to the exemplary embodiments. The exemplary embodiments may also be configured as a program that causes a computer to execute the functions of each component provided in the impression measurement device. The exemplary embodiments may also be configured as a non-transitory computer readable storage medium storing the program.

Otherwise, the configuration of the impression measurement device described in the exemplary embodiments above is one example, and may be modified according to circumstances within a range that does not depart from the gist.

Also, the process flow of the program described in the exemplary embodiments above is one example, and unnecessary steps may be removed, new steps may be added, or the processing sequence may be rearranged within a range that does not depart from the gist.

Also, the exemplary embodiments above describe a case in which the process according to the exemplary embodiments is realized by a software configuration using a computer by executing a program, but the configuration is not limited thereto. The exemplary embodiments may also be realized by a hardware configuration, or by a combination of a hardware configuration and a software configuration, for example.

The foregoing description of the exemplary embodiments of the present disclosure has been provided for the purposes of illustration and description. It is not intended to be exhaustive or to limit the disclosure to the precise forms disclosed. Obviously, many modifications and variations will be apparent to practitioners skilled in the art. The embodiments were chosen and described in order to best explain the principles of the disclosure and its practical applications, thereby enabling others skilled in the art to understand the disclosure for various embodiments and with the various modifications as are suited to the particular use contemplated. It is intended that the scope of the disclosure be defined by the following claims and their equivalents.

What is claimed is:

1. An impression measurement device comprising:
an acquisition unit that, in a case in which a user interviews a plurality of persons in an order, acquires biological information of the user during the interview with each of the plurality of persons;
a setting unit that, in a case of deciding weight values assigned to the biological information acquired by the acquisition unit for each of the plurality of persons, sets larger weight values for later persons compared to earlier persons in the order; and
a correction unit that uses the weight values set by the setting unit to correct the biological information of the user with respect to each of the plurality of persons.

2. The impression measurement device according to claim 1, wherein
an interview time per person is associated with the biological information in advance.

3. The impression measurement device according to claim 1, wherein
the biological information is expressed by a relationship between each of the plurality of persons and a total power that is a combined total of a component that varies at a high frequency corresponding to respiratory variation and a component that varies at a low frequency corresponding to blood pressure variation.

4. The impression measurement device according to claim 3, wherein
the setting unit sets the weight values with respect to an activity level of the total power, and
the correction unit corrects the biological information such that the activity level becomes larger.

5. The impression measurement device according to claim 3, wherein
the setting unit sets the weight values with respect to a response time of the total power, and
the correction unit corrects the biological information such that the response time becomes shorter.

6. The impression measurement device according to claim 3, wherein
the setting unit sets the weight values with respect to each of an activity level of the total power and a response time of the total power, and
the correction unit corrects the biological information such that the activity level becomes larger and the response time becomes shorter.

7. The impression measurement device according to claim 1, further comprising:
a derivation unit that, on a basis of the biological information corrected by the correction unit, assigns a slope score that is a score with respect to a slope of a graph indicating the biological information and an interview order score that is a score with respect to the order of interviews to each of the plurality of persons, and derives a total score for each of the plurality of persons from the slope score and the interview order score.

8. The impression measurement device according to claim 7, wherein
the derivation unit additionally uses behavior information obtained from a behavior of the user to additionally assign a behavior score that is a score with respect to the behavior information to each of the plurality of persons, and additionally derives a total score for each of the plurality of persons from the slope score, the interview order score, and the behavior score.

9. The impression measurement device according to claim 7, further comprising:
a control unit that controls a display of a rank table that ranks each of the plurality of persons on a basis of the total score for each of the plurality of persons derived by the derivation unit.

10. The impression measurement device according to claim 7, wherein
the slope score is assigned a larger value to an extent that the slope of the graph is larger in a positive direction, and
the interview order score is assigned a larger value for partners later in the order of interviews.

11. The impression measurement device according to claim 1, wherein
the setting unit sets the weight values on a basis of biological information corresponding to each of a common behavior performed before and after the interviews, and the order.

12. The impression measurement device according to claim 11, wherein
the biological information is expressed by a total power that is a combined total of a component that varies at a high frequency corresponding to respiratory variation and a component that varies at a low frequency corresponding to blood pressure variation, and
the weight values are values obtained by dividing an average value of a difference between the total power in the common behavior before the interviews and the total power in the common behavior after the interviews by the number of the plurality of persons, and multiplying by a value expressing the order.

13. The impression measurement device according to claim 11, wherein
the biological information is expressed by a total power that is a combined total of a component that varies at a high frequency corresponding to respiratory variation and a component that varies at a low frequency corresponding to blood pressure variation, and
the weight values are obtained by taking a compression ratio obtained by dividing a number of data points for the total power in the common behavior before the interviews by a number of data points for the total power in the common behavior after the interviews, dividing by the number of the plurality of persons, and multiplying by a value expressing the order.

14. A non-transitory computer readable medium storing a program causing a computer to execute a process for functioning as the impression measurement device according to claim 1.

15. An impression measurement device comprising:
acquiring means for, in a case in which a user interviews a plurality of persons in an order, acquiring biological information of the user during the interview with each of the plurality of persons;
setting means for, in a case of deciding weight values assigned to the biological information acquired by the acquiring means for each of the plurality of persons, setting larger weight values for later persons compared to earlier persons in the order; and
correcting means for using the weight values set by the setting means to correct the biological information of the user with respect to each of the plurality of persons.
